(19) United States
(12) Patent Application Publication
(10) Pub. No.: US 2026/0341526 A1
(43) Pub. Date: Oct. 29, 2026

(54) CLINICAL PREDICTIONS BASED ON LONGITUDINAL MULTI-MODAL MEDICAL DATA

(71) Applicant: Cornell University, Ithaca, NY (US)

(72) Inventors: Mert Sabuncu, Princeton, NJ (US); Batuhan Karaman, New York, NY (US)

(21) Appl. No.: 19/657,976

(22) Filed: Apr. 24, 2026

Related U.S. Application Data
(60) Provisional application No. 63/794,028, filed on Apr. 24, 2025.
(60) Provisional application No. 64/047,532, filed on Apr. 23, 2026.

(51) Int. Cl. G16H 50/30; G16H 10/40; G16H 10/60; G16H 30/40; G16H 50/20

(52) U.S. Cl. CPC G16H 50/30; G16H 10/40; G16H 10/60; G16H 30/40; G16H 50/20

(57) ABSTRACT

Method for disease detection and future risk prediction based on longitudinal multi-modal medical data is described herein. Longitudinal data processing component of longitudinal risk neural network receives multi-modal medical data from a plurality of modalities associated with patient visits over time. Multi-modal medical data is encoded to generate encoded medical data, and a plurality of visit embeddings is generated based on the encoded medical data of each patient visit. The visit embeddings are processed to capture temporal relationships between the visit embeddings and to generate a history embedding representing the patient's longitudinal record. A prediction component of the longitudinal risk neural network generates a clinical prediction by determining a baseline risk from the history embedding, computing a plurality of incremental risk outputs for a plurality of future time intervals, and generating a cumulative risk prediction by combining the baseline risk with a sum of the incremental non-negative risk outputs.

### CROSS-REFERENCE TO RELATED APPLICATIONS

This application claims the benefit of priority to U.S. Provisional Patent Application No. 63/794,028, filed Apr. 24, 2025, and to U.S. Provisional Patent Application No. 64/047,532, filed Apr. 23, 2026, the entire contents of which are incorporated herein by reference.

### GOVERNMENT SUPPORT CLAUSE

This invention was made with government support under AG053949, and MH130899 awarded by the National Institutes of Health, and 1748377 awarded by the National Science Foundation. The government has certain rights in the invention.

### BACKGROUND

Artificial Intelligence (AI)-based diagnostic tools have become increasingly important in modern healthcare. However, existing technologies and solutions have several key limitations. For example, many existing AI systems rely primarily on single-timepoint data, such as individual imaging scans or one-off lab readings, when assessing a patient's health status. This limited temporal scope can result in suboptimal early detection and less accurate predictions of disease progression, as meaningful patterns often emerge over multiple visits. Existing approaches further lack mechanisms to enforce clinically consistent temporal behavior in predicted risk trajectories, such as monotonic increases in cumulative disease risk over time. Additionally, conventional systems do not distinguish between inherently missing data modalities and artificially masked modalities during training, leading to reduced robustness in real-world clinical settings.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a block diagram illustrating a networked system 100 in which the present disclosure may be deployed, according to some examples.

FIG. 2 illustrates a block diagram of the longitudinal risk neural network 200, in accordance with some examples.

FIG. 3 illustrates a block diagram of the longitudinal data processing component 202, in accordance with some examples.

FIG. 4 illustrates a block diagram of the longitudinal data processing component 202, in accordance with some examples.

FIG. 5 illustrates a process 500 for clinical predictions based on longitudinal medical data analysis, in accordance with some examples.

FIG. 6 is a flowchart illustrating sub-operations of operation 510 of the process 500 described with reference to FIG. 5, in accordance with some examples. I

FIG. 7 is a block diagram showing a software architecture within which the present disclosure may be implemented, in accordance with some examples.

FIG. 8 is a diagrammatic representation of a machine, in the form of a computer system within which a set of instructions may be executed for causing the machine to perform any one or more of the methodologies discussed herein in accordance with some examples.

### DETAILED DESCRIPTION

In the example of breast cancer screening specifically, current approaches predominantly analyze only the present mammogram, possibly together with limited prior visit data for a patient. This approach fails to take advantage of the comprehensive longitudinal mammographic history available.

Examples in the disclosure are directed to a system and method for disease detection and future risk prediction based on longitudinal multi-modal medical data. The system and method as described in the examples provide several significant technical advantages over conventional single-timepoint AI diagnostic systems through a novel two-stage aggregation architecture and temporal processing capabilities. In particular, the system introduces a constrained cumulative risk formulation that enforces monotonicity across future prediction horizons, and a modality-aware missing data handling mechanism that distinguishes between observed and training-induced missingness.

In some examples, the system effectively integrates data from multiple visits into a unified, time-aware artificial intelligence (AI) architecture and is capable of capturing longitudinal changes across various data types including changes in imaging features, blood biomarkers, and textual clinical notes and fusing these changes over time to generate accurate predictions for multiple clinical tasks.

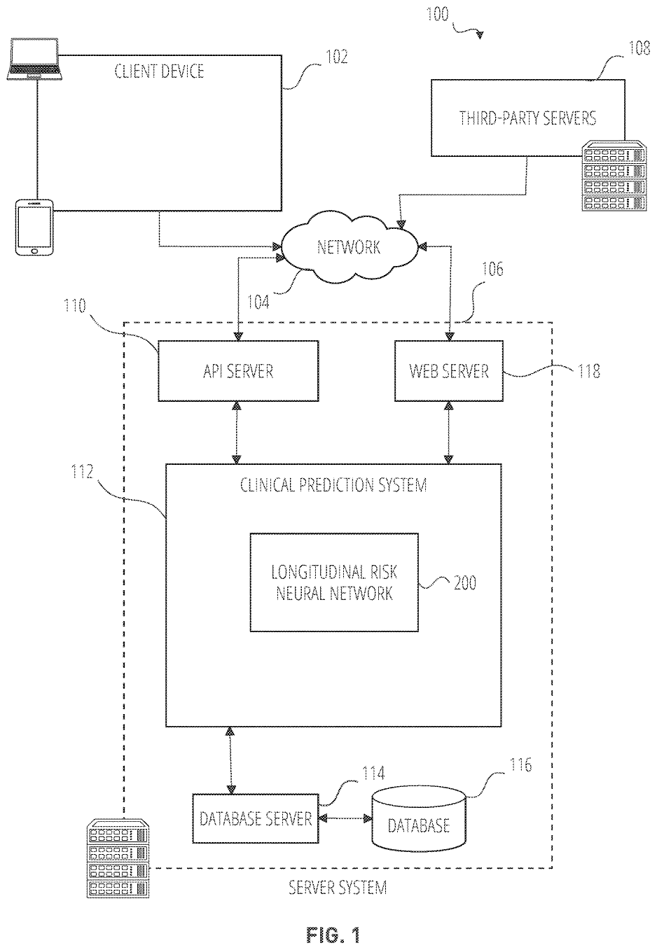

FIG. 1 is a block diagram showing an example networked system 100 for exchanging data (e.g., medical data, clinical prediction, etc.) over a network. The networked system 100 can includes multiple instances of a client device 102 that is communicatively coupled to a server system 106 and third-party servers 108 via a network 104 (e.g., the Internet, Intranet).

A client device 102 can be associated with a patient, a medical professional, a hospital, etc. The client device 102 is able to communicate and exchange data the server system 106 via the network 104. The data exchanged between the client device 102 and the server system 106 can include the medical data from a plurality of modalities associated with a plurality of patient visits over time, the clinical predictions, etc.

Turning now specifically to the server system 106, an Application Program Interface (API) server 110 is coupled to, and provides a programmatic interface to, clinical prediction system 112. The clinical prediction system 112 are communicatively coupled to a database server 114, which facilitates access to a database 116 that stores data associated with data processed by the clinical prediction system 112. Similarly, a web server 118 is coupled to the clinical prediction system 112, and provides web-based interfaces to the clinical prediction system 112. To this end, the web server 118 processes incoming network requests over the Hypertext Transfer Protocol (HTTP) and several other related protocols.

The Application Program Interface (API) server 110 receives and transmits data between the client device 102 and the clinical prediction system 112. The clinical prediction system 112 includes a longitudinal risk neural network 200.

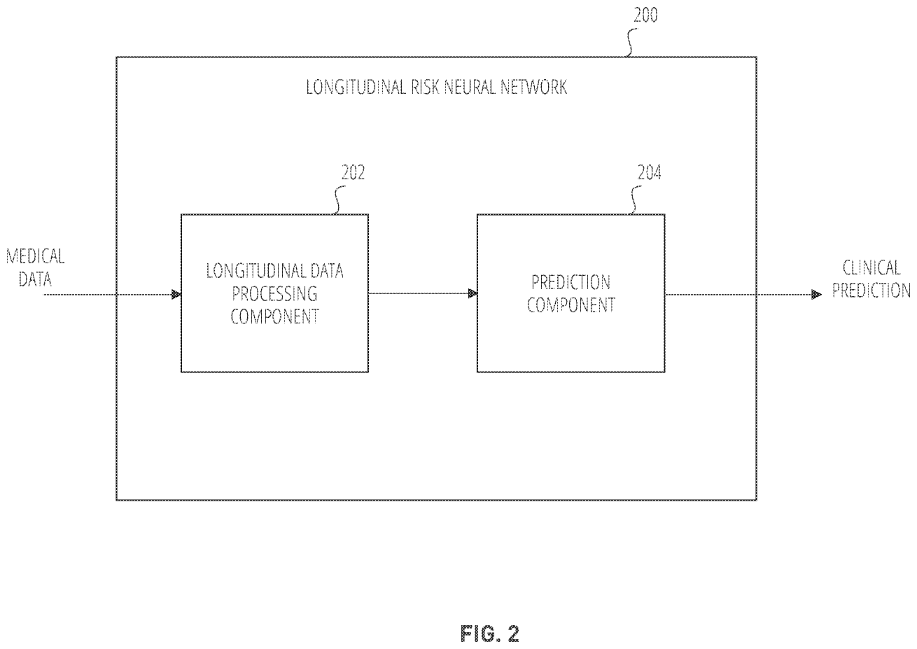

FIG. 2 illustrates a block diagram of the longitudinal risk neural network 200 of the clinical prediction system 112, in accordance with some examples. The longitudinal risk neural network 200 may be implemented as part of a clinical prediction system, such as the clinical prediction system 112 described with reference to FIG. 1, and may be executed by one or more processors of a server system, a client device, or a combination thereof.

As shown in FIG. 2, the longitudinal risk neural network 200 comprises a longitudinal data processing component 202 and a prediction component 204. The longitudinal data processing component 202 is coupled to the prediction component 204 such that an output of the longitudinal data processing component 202 is provided as an input to the prediction component 204.

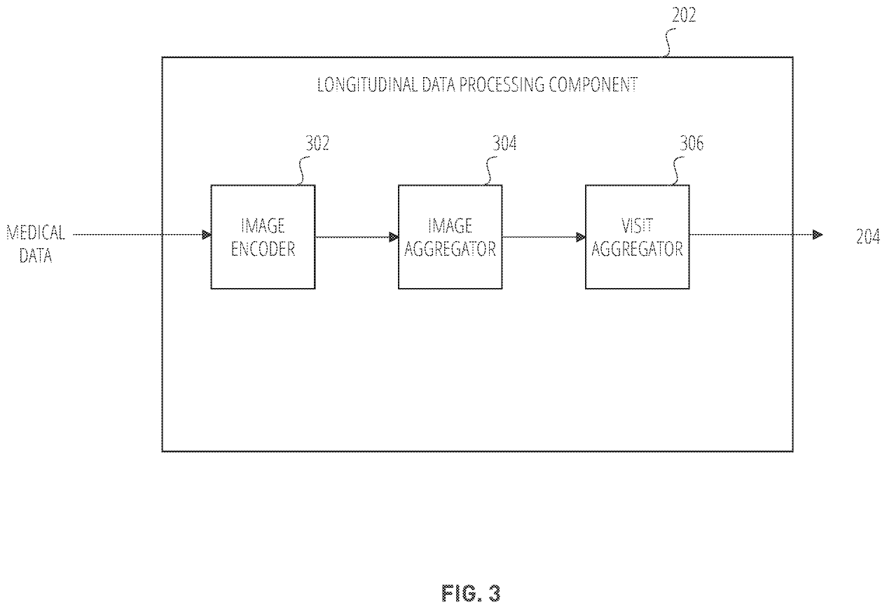

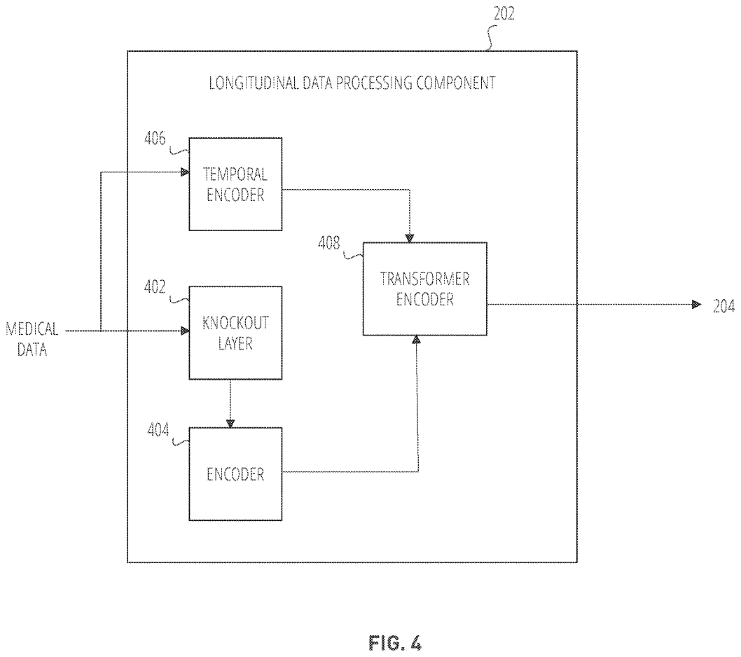

The longitudinal data processing component 202 receives the medical data from a plurality of modalities associated with a plurality of patient visits of a patient. In some embodiments, the medical data includes data from two or more modalities, such as medical imaging data, laboratory values, clinical test results, clinical notes, waveform data, genetic data, cognitive assessment data, cerebrospinal fluid (CSF) biomarker data, risk factor metadata, demographic data, or any combination thereof. The longitudinal data processing component 202 encodes the medical data to generate encoded medical data, generates a plurality of visit embeddings based on the encoded medical data of each patient visit, and processes the plurality of visit embeddings to capture temporal relationships between the visit embeddings. The longitudinal data processing component 202 outputs a history embedding representing a longitudinal record for the patient. Further details of the longitudinal data processing component 202 are described below with reference to FIG. 3 and FIG. 4.

The prediction component 204 receives the history embedding from the longitudinal data processing component 202 and generates the clinical prediction based on the history embedding. In some embodiments, the prediction component 204 determines a baseline risk using the history embedding, computes a plurality of incremental risk outputs for each of a plurality of future time intervals using the history embedding, and generates a cumulative risk prediction for a selected future time interval by combining the baseline risk with a sum of the incremental risk outputs associated with a current time interval through the incremental risk outputs associated with the selected future time interval. In some embodiments, the incremental risk outputs are computed using linear layers followed by Rectified Linear Unit (ReLU) activations, such that the incremental risk outputs are constrained to be non-negative and the cumulative risk prediction is monotonically non-decreasing across the plurality of future time intervals.

In some embodiments, the clinical prediction comprises a likelihood of disease, a disease risk score, a categorization of a condition, a predicted clinical variable value, a multi-label prediction, or any combination thereof. By way of non-limiting example, the clinical prediction may comprise a likelihood of breast cancer, a breast cancer risk score, a likelihood of progression from a cognitively normal state to a mild cognitive impairment state, a likelihood of progression from a mild cognitive impairment state to an Alzheimer's disease state, or any combination thereof.

In some embodiments, the prediction component 204 comprises an additive hazard layer configured to generate the clinical prediction based on the history embedding. By way of non-limiting example, in a breast cancer prediction embodiment, the prediction component 204 leverages the history embedding, denoted as m, to predict future breast cancer risk. The prediction component 204 determines a baseline risk B(m) using the history embedding and computes, for each of a plurality of future time intervals, an incremental risk output Hᵢ(m) using one or more linear layers applied to the history embedding. The incremental risk outputs Hᵢ(m) are constrained to be non-negative, e.g., by passing through Rectified Linear Unit (ReLU) activations. The prediction component 204 then generates a cumulative k-year risk prediction, denoted P(t_{cancer}=k|m), by combining the baseline risk B(m) with a sum of the incremental risk outputs Hᵢ(m) from i=1 through i=k, and applying a sigmoid function σ to the combination, as expressed by Equation:

$$P ( {t}_{c a n c e r} = k \vert m ) = σ ( B ( m ) + {\sum }_{i = 1}^{k} {H}_{i} ( m ) )$$

In the Equation above, m denotes the history embedding representing the patient's longitudinal record, B(m) denotes the baseline risk computed from the history embedding, Hᵢ(m) denotes the non-negative incremental risk output associated with the i-th future time interval, k denotes the selected future time interval (e.g., a selected future year), and σ denotes the sigmoid function. Because the incremental risk outputs Hᵢ(m) are constrained to be non-negative, the cumulative risk prediction P(t_{cancer}=k|m) is monotonically non-decreasing as k increases, thereby guaranteeing that the predicted risk grows, or remains constant, over time. In some embodiments, the cumulative k-year risk prediction represents a likelihood that the patient will be diagnosed with breast cancer within k years following a current time interval (e.g., a time interval corresponding to a most recent patient visit). In some embodiments, k is selected from a range of one to five years, although other ranges may be used without departing from the scope of the present disclosure.

In some embodiments, the prediction component 204 comprises one or more prediction components, each of which comprises an additive hazard layer configured to generate the clinical prediction based on the history embedding. By way of non-limiting example, in an Alzheimer's disease prediction embodiment, the prediction component 204 leverages the history embedding, denoted as m, to estimate the risk of disease progression. The prediction component 204 determines a baseline risk B(m) using the history embedding and computes, for each of a plurality of future time intervals, an incremental risk output Hᵢ(m) using one or more linear layers applied to the history embedding. The incremental risk outputs Hᵢ(m) are passed through Rectified Linear Unit (ReLU) activations to ensure non-negative hazard values. The prediction component 204 then generates a cumulative k-year progression risk prediction, denoted P(t_{progression}=k|m), by combining the baseline risk B(m) with a sum of the incremental risk outputs Hᵢ(m) from i=1 through i=k, and applying a sigmoid function σ to the combination, as expressed by Equation:

$$P ( {t}_{progression} = k \vert m ) = σ ( B ( m ) + {\sum }_{i = 1}^{k} {H}_{i} ( m ) )$$

For k∈{1, 2, . . . , 5}, where m denotes the history embedding representing the patient's longitudinal record, B(m) denotes the baseline risk computed from the history embedding, Hᵢ(m) denotes the incremental risk output associated with the i-th future time interval, k denotes the selected future time interval, and σ denotes the sigmoid function. Because the incremental risk outputs Hᵢ(m) are constrained to be non-negative, the cumulative progression risk P(t_{progression}=k|m) is monotonically non-decreasing as k increases, thereby ensuring that the predicted risk grows, or remains constant, over time.

In some embodiments, the prediction component 204 comprises a first prediction component for predicting a first level of progression and a second prediction component for predicting a second level of progression. By way of non-limiting example, the prediction component 204 may comprise a first prediction component configured to predict a likelihood of progression from a cognitively normal (CN) state to a mild cognitive impairment (MCI) state, and a second prediction component configured to predict a likelihood of progression from the MCI state to an Alzheimer's disease (AD) state. In some embodiments, the prediction component 204 selects one of the plurality of survival heads based on a current diagnosis of the patient at a prediction time. For example, where the current diagnosis of the patient is the CN state, the prediction component 204 generates the clinical prediction using the first prediction component, and where the current diagnosis of the patient is the MCI state, the prediction component 204 generates the clinical prediction using the second prediction component.

In some embodiments, the plurality of prediction components share a common backbone of the longitudinal risk neural network 200, such as a transformer-based backbone of the longitudinal data processing component 202. The shared backbone allows the longitudinal risk neural network 200 to be trained as a single unified network on training data incorporating labels from a plurality of follow-up years, while generating progression predictions across multiple future time horizons without requiring separate models for each time horizon. This arrangement enables the longitudinal risk neural network 200 to learn a representation of disease progression dynamics applicable to multiple disease stage transitions and multiple future time intervals.

FIG. 3 is a block diagram illustrating the longitudinal data processing component 202 of the longitudinal risk neural network 200 in accordance with some examples. In some embodiments, the longitudinal data processing component 202 shown in FIG. 3 is configured to process medical data comprising medical imaging data associated with a plurality of patient visits of a patient.

As shown in FIG. 3, the longitudinal data processing component 202 comprises an image encoder 302, an image aggregator 304, and a visit aggregator 306. The image encoder 302 is coupled to the image aggregator 304, and the image aggregator 304 is coupled to the visit aggregator 306. An output of the visit aggregator 306 is provided to the prediction component 204 described with reference to FIG. 2.

The image encoder 302 receives the medical data associated with the plurality of patient visits and processes the medical data to generate a plurality of image embeddings. In some embodiments, the medical data for each patient visit of the plurality of patient visits comprises one or more medical images. By way of non-limiting example, in a breast cancer prediction embodiment, the medical data for each patient visit comprises four two-dimensional mammographic images, including a craniocaudal (CC) view and a mediolateral oblique (MLO) view for each of a left breast and a right breast of the patient. Other medical imaging modalities, such as ultrasound images, X-ray images, magnetic resonance imaging (MRI) images, computed tomography (CT) scans, or any combination thereof, may be processed by the image encoder 302 without departing from the scope of the present disclosure.

In some embodiments, the image encoder 302 comprises a convolutional neural network (CNN). By way of non-limiting example, the image encoder 302 may comprise a Residual Network (ResNet)-based CNN, such as a ResNet-18 backbone, followed by a pooling layer to produce one-dimensional image embeddings. The image encoder 302 applies shared weights across the images within a patient visit and across patient visits, such that distinct image embeddings are produced for each image. In some embodiments, parameters of the image encoder 302 are pre-trained on a separate dataset and subsequently frozen during training of other components of the longitudinal risk neural network 200.

The image aggregator 304 receives the plurality of image embeddings generated by the image encoder 302 and combines the image embeddings associated with each patient visit to generate a plurality of visit embeddings. Each visit embedding of the plurality of visit embeddings corresponds to a respective patient visit and integrates information across the images associated with that patient visit. In some embodiments, the image aggregator 304 comprises a transformer encoder. The image aggregator 304 may condition each image embedding on one or more attributes of the corresponding image, such as a view (e.g., CC or MLO) and a laterality (e.g., left or right), using learned positional embeddings. By way of non-limiting example, an affine transformation may be applied to each image embedding x according to the following expression:

h=(W_{scale}e)⊙x+(W_{shift}e),

where e denotes a positional embedding associated with the view and laterality of the image, W_{scale} and W_{shift} denote parameter matrices and are two-dimensional and fixed across all views and literalities, ensuring uniform scaling and shifting of the embeddings, and ⊙ denotes element-wise multiplication (e.g., dot product). Following the conditioning, the image aggregator 304 processes the conditioned image embeddings using a self-attention block and applies attention pooling to produce the visit embedding for the patient visit. By way of non-limiting example, in some embodiments, the attention pooling is implemented using a linear layer followed by a softmax activation.

The visit aggregator 306 receives the plurality of visit embeddings generated by the image aggregator 304 and processes the plurality of visit embeddings to capture temporal relationships between the visit embeddings and to generate the history embedding representing the longitudinal record of the patient. In some embodiments, the visit aggregator 306 comprises a transformer encoder configured to process each visit embedding of the plurality of visit embeddings as an individual token of an input sequence. The visit aggregator 306 may incorporate temporal encodings associated with each visit embedding to enable the visit aggregator 306 to interpret the temporal ordering of the patient visits. By way of non-limiting example, the temporal encodings may comprise fixed sine and cosine positional encodings as temporal encodings.

The visit aggregator 306 processes the input sequence of visit embeddings using one or more self-attention blocks to capture both short-term and long-term dependencies among the plurality of patient visits. The use of temporal encodings tied to visit timing enables the model to process irregularly spaced longitudinal data without requiring uniform sampling intervals. Following the one or more self-attention blocks, the visit aggregator 306 applies a sequence pooling operation to aggregate information across the visit embeddings into a fixed-length history embedding. In some embodiments, the sequence pooling operation comprises average sequence pooling along a token axis of the input sequence. The history embedding generated by the visit aggregator 306 is provided to the prediction component 204 for generating the clinical prediction, as described with reference to FIG. 2.

In some embodiments, the longitudinal data processing component 202 is configured to process an arbitrary number of patient visits, such that the history embedding may be generated from medical data associated with one patient visit, two patient visits, three patient visits, four patient visits, five patient visits, or more patient visits. In some embodiments, the longitudinal data processing component 202 is further configured to process patient visits occurring at irregular time intervals, such that the plurality of patient visits need not be separated by uniform time intervals.

In accordance with some other examples, FIG. 4 illustrates a block diagram of the longitudinal data processing component 202 of the longitudinal risk neural network 200. In some embodiments, the longitudinal data processing component 202 shown in FIG. 4 is configured to process medical data from a plurality of modalities, such as cognitive assessments, magnetic resonance imaging (MRI) volumetric measurements, cerebrospinal fluid (CSF) biomarkers, genetic data, demographic data, or any combination thereof.

As shown in FIG. 4, the longitudinal data processing component 202 comprises a knockout layer 402, an encoder 404, a temporal encoder 406, and a transformer encoder 408. The longitudinal data processing component 202 receives medical data associated with a plurality of patient visits of a patient over time.

The knockout layer 402 receives the multi-modal medical data and processes the multi-modal medical data, for each of the patient visits, to identify missing features or modalities within the medical data. The knockout layer 402 can also process the multi-modal medical data, for each of the patient visits, while identifying missing features or modalities within the medical data. The knockout layer 402 assigns out-of-distribution values or highly improbable placeholder values to the identified missing features. The “highly improbable values” can refer to values that lie outside or at the extreme tails of a distribution observed in data, such that the values are unlikely to occur under normal data-generating conditions. By way of non-limiting example, the out-of-distribution or highly improbable values may comprise a first designated numerical value (e.g., +10) to encode missing features introduced during training and a second designated numerical value (e.g., −10) to encode missing features present in the received medical data. The knockout layer 402 outputs the medical data with the assigned out-of-distribution or highly improbable values for subsequent processing, enabling the longitudinal data processing component 202 to process patient visits having incomplete data modalities. In some embodiments, the use of distinct placeholder values for observed missingness and training-induced missingness enables the model to differentiate between structurally absent data and simulated modality dropout, thereby improving generalization to heterogeneous real-world data.

In some embodiments, the knockout layer 402 operates in two distinct modes. During inference, the knockout layer 402 identifies features that are missing from the received medical data for a given patient visit and assigns out-of-distribution or highly improbable placeholder values to the identified missing features, thereby enabling the longitudinal risk neural network 200 to process patient visits having incomplete data modalities without requiring imputation. During training, the knockout layer 402 additionally operates as a data augmentation mechanism by randomly replacing one or more data modalities of one or more patient visits with out-of-distribution or highly improbable placeholder values at a specified knockout rate, even where the corresponding features are present in the training data, thereby simulating missingness patterns encountered at inference time. In some embodiments, observed missingness and knockout-induced missingness are encoded using distinct placeholder values, such that the longitudinal risk neural network 200 may distinguish between features that are inherently missing from the received medical data and features that have been masked during training. The two modes of operation enable the longitudinal risk neural network 200 to generalize across incomplete or heterogeneous data modalities at inference time while being trained on data samples that may include partially or fully observed modalities. This dual-mode missingness encoding differs from conventional imputation-based approaches by preserving missingness as an informative signal rather than reconstructing missing values.

The encoder 404 receives the output from the knockout layer 402 and encodes the medical data to generate encoded medical data. In some embodiments, the encoder 404 comprises a shared linear layer that applies a linear projection to the medical data of each patient visit to generate a plurality of individual tokens, such that each patient visit is encoded as an individual token.

The temporal encoder 406 encodes temporal ordering of the plurality of patient visits by incorporating temporal encoding vectors within each of the individual tokens. The temporal encoding vectors are based on a time interval between a data collection time of a patient visit and a target future prediction time. In some embodiments, the temporal encoder 406 projects a timestamp of each patient visit into a latent space using a linear transformation. By way of non-limiting example, the timestamp may be measured in months, where a present time is set to zero and past visits are represented by negative values. The temporal encoder 406 sums the projected visit embeddings and the temporal encodings to generate temporally-encoded visit embeddings.

In some embodiments, the temporal encoding applied to the plurality of visit embeddings may be implemented using one or more alternative approaches. In a first embodiment, the temporal encoding comprises fixed sinusoidal positional encodings, wherein sine and cosine functions of varying frequencies are used to encode the temporal position of each patient visit within the input sequence, as described with reference to the visit aggregator 306 of FIG. 3. In a second embodiment, the temporal encoding comprises a learned linear projection, wherein a timestamp associated with each patient visit is projected into a latent space using a linear transformation and summed with the corresponding visit embedding to produce a temporally-encoded visit embedding, as described with reference to the temporal encoder 406 of FIG. 4. The first embodiment and the second embodiment are alternative embodiments, and either approach may be used without departing from the scope of the present disclosure. In some embodiments, the temporal encoding is based on a time interval between a data collection time of a patient visit and a target future prediction time, while in other embodiments the temporal encoding is based on an absolute or relative timestamp of the patient visit. Other temporal encoding approaches, including learned absolute positional encodings, learned relative positional encodings, or combinations of sinusoidal and learned components, may also be used.

The transformer encoder 408 receives the temporally-encoded visit embeddings and processes the plurality of individual tokens using self-attention mechanisms to capture short-term and long-term dependencies among the plurality of patient visits. By way of non-limiting example, a sequence pooling layer then applies average pooling along a token axis, aggregating information from the individual tokens into a fixed-length representation. This fixed-length representation comprises the history embedding representing the longitudinal record for the patient. The history embedding is provided to the prediction component 204 for generating the clinical prediction, as described with reference to FIG. 2.

In some embodiments, the longitudinal risk neural network 200 is trained end-to-end using labeled longitudinal data from a plurality of patients. Each training sample comprises medical data from one or more patient visits spanning a longitudinal history window, together with one or more ground-truth labels corresponding to one or more future time intervals following a reference visit. Ground-truth labels may comprise diagnostic codes, measured clinical values, time-to-event outcomes, or any combination thereof. In some embodiments, training samples are generated by treating each patient visit within a training dataset as a reference time point, constructing a longitudinal history sequence from one or more patient visits occurring prior to the reference time point up to a specified maximum history duration, and associating a plurality of ground-truth labels corresponding to a plurality of future time intervals subsequent to the reference time point. Data splits are maintained at the individual subject level to prevent information leakage between training, validation, and test sets.

In some embodiments, the longitudinal risk neural network 200 is trained using a loss function selected based on the clinical prediction task, including a cross-entropy loss for classification tasks, a mean-squared-error loss for regression tasks, or a survival loss for time-to-event tasks. In some embodiments, a re-weighted cross-entropy loss is used to address class imbalance across the plurality of future time intervals, wherein sample-specific weights are assigned based on class frequencies. In some embodiments, one or more components of the longitudinal data processing component 202 are pre-trained on a unimodal dataset and subsequently used with frozen parameters during training of the remaining components, or the longitudinal risk neural network 200 is trained end-to-end without freezing any components.

In some embodiments, the longitudinal risk neural network 200 is trained using a longitudinal data augmentation strategy in which one or more prior patient visits are randomly omitted from a longitudinal history sequence during training according to a specified augmentation probability. In some embodiments, the knockout layer 402 operates during training to randomly replace one or more data modalities within a patient visit with out-of-distribution or highly improbable placeholder values at a specified knockout rate, and observed missingness (e.g., missing features) and knockout-induced missingness (e.g., missing features) are encoded using distinct placeholder values. The longitudinal data augmentation and modality-based knockout enable the longitudinal risk neural network 200 to generalize across variable longitudinal history durations, variable data collection frequencies, and incomplete data modalities at inference time.

In some embodiments, the longitudinal risk neural network 200 is trained using mini-batch stochastic gradient descent with an Adam optimizer, and training is regularized using one or more of dropout, L2 regularization, and early stopping based on a validation loss. Architectural hyperparameters, including an embedding dimension, a number of self-attention heads, and a number of self-attention layers, may be selected based on validation performance using a hyperparameter optimization procedure such as a grid search.

### Process for Clinical Predictions Based on Longitudinal Medical Data Analysis

Although the described flowcharts can show operations as a sequential process, many of the operations can be performed in parallel or concurrently. In addition, the order of the operations may be re-arranged. A process is terminated when its operations are completed. A process may correspond to a method, a procedure, an algorithm, etc. The operations of methods may be performed in whole or in part, may be performed in conjunction with some or all of the operations in other methods, and may be performed by any number of different systems, such as the systems described herein, or any portion thereof, such as a processor included in any of the systems.

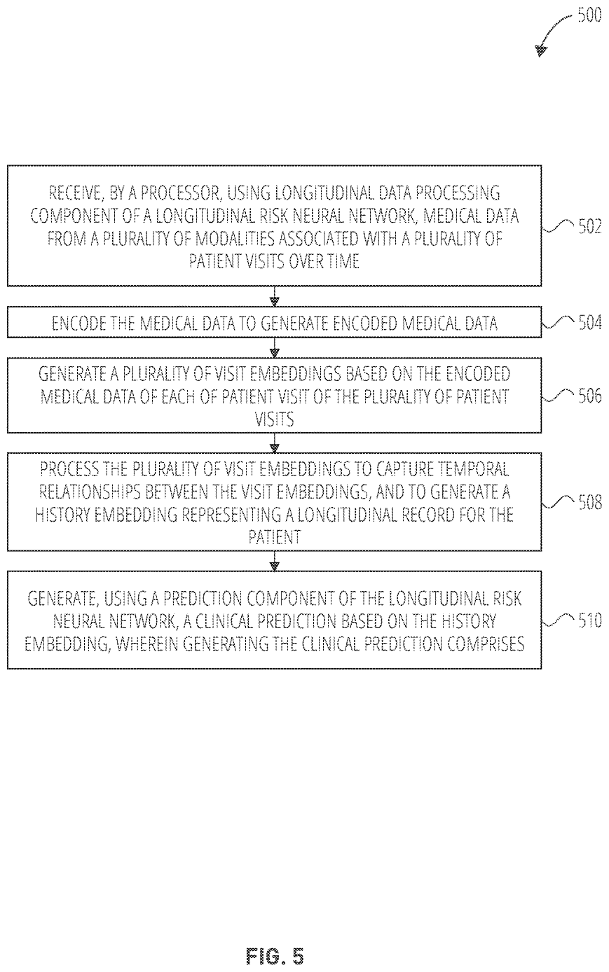

FIG. 5 illustrates a process 500 for clinical predictions based on longitudinal medical data analysis, in accordance with some examples. In some embodiments, the process 500 is performed by one or more processors of a clinical prediction system, such as the clinical prediction system 112 described with reference to FIG. 1, executing the longitudinal risk neural network 200 described with reference to FIG. 2.

At operation 502, a processor receives, using the longitudinal data processing component 202 of the longitudinal risk neural network 200, medical data from a plurality of modalities associated with a plurality of patient visits over time. The patient visits are associated with a patient. In some embodiments, the medical data from the plurality of modalities comprises medical imaging data, risk factor metadata, laboratory values, clinical test results, clinical notes, waveform data, genetic data, other data types, or any combination thereof. By way of non-limiting example, the medical imaging data may comprise mammograms, ultrasound images, X-ray images, Magnetic Resonance Imaging (MRI) images, computed tomography (CT) scans, or any combination thereof. The risk factor metadata may comprise age, family history, body mass index (BMI), hormonal factors, or any combination thereof.

In some embodiments, the medical data from the plurality of modalities comprises cognitive assessments, cerebrospinal fluid (CSF) biomarkers, MRI volumetric measurements, genetic data, demographic data, or any combination thereof. By way of non-limiting example, the demographic data may comprise age, sex, education, marital status, race, ethnicity, or any combination thereof. In some embodiments, the cognitive assessments comprise one or more of a Clinical Dementia Rating (CDR), a Mini-Mental State Examination (MMSE), an Alzheimer's Disease Assessment Scale (ADAS-Cog), a Montreal Cognitive Assessment (MoCA), a Functional Activities Questionnaire (FAQ), an Everyday Cognition (ECog) assessment, a Rey Auditory Verbal Learning Test, a Logical Memory Delayed Recall, a Trail Making Test Part B, a Digit Symbol Substitution test, or any combination thereof. In some embodiments, the CSF biomarkers comprise one or more of Amyloid-Beta 1-42, Total Tau (T-Tau), Phosphorylated Tau (P-Tau), or any combination thereof. In some embodiments, the MRI volumetric measurements comprise one or more measurements of ventricles, hippocampus, whole brain, entorhinal cortex, fusiform cortex, middle temporal cortex, intracranial volume (ICV), or any combination thereof.

In some embodiments, the medical data is received from a plurality of patient visits occurring at regular time intervals (e.g., annual visits, biennial visits) or at irregular time intervals. The longitudinal data processing component 202 is configured to process an arbitrary number of patient visits, such that the plurality of patient visits may comprise one patient visit, two patient visits, three patient visits, four patient visits, five patient visits, or more patient visits.

At operation 504, the processor encodes the medical data to generate encoded medical data. In some embodiments, encoding the medical data comprises processing, by an image encoder 302 of the longitudinal data processing component 202, the medical data to generate a plurality of image embeddings associated with the plurality of patient visits. In such embodiments, the encoded medical data of each patient visit of the plurality of patient visits comprises one or more of the plurality of image embeddings associated with that patient visit. By way of non-limiting example, the image encoder 302 may comprise a Residual Network (ResNet)-based convolutional neural network (CNN), such as a ResNet-18 backbone followed by a global max pooling layer.

In some embodiments, encoding the medical data further comprises processing, by a knockout layer 402, for each of the patient visits, the medical data to identify missing features, and assigning, by the knockout layer 402, out-of-distribution or highly improbable values to the missing features. By way of non-limiting example, the out-of-distribution or highly improbable values may comprise a first designated numerical value to encode missing features introduced during training and a second designated numerical value to encode missing features present in the received medical data.

At operation 506, the processor generates a plurality of visit embeddings based on the encoded medical data of each patient visit of the plurality of patient visits. In some embodiments, generating the plurality of visit embeddings comprises combining, by an image aggregator 304 of the longitudinal data processing component 202, the plurality of image embeddings to generate the plurality of visit embeddings. In some embodiments, the image aggregator 304 comprises a transformer encoder and applies attention-based pooling to combine image embeddings corresponding to a common patient visit into a single visit embedding.

In some embodiments, generating the plurality of visit embeddings comprises encoding temporal ordering of the plurality of patient visits by incorporating temporal encoding vectors within each of a plurality of individual tokens, wherein the plurality of patient visits are encoded as the plurality of individual tokens. The temporal encoding vectors are based on a time interval between a patient visit's data collection and a target future prediction time.

At operation 508, the processor processes the plurality of visit embeddings to capture temporal relationships between the visit embeddings and to generate a history embedding representing a longitudinal record for the patient. In some embodiments, processing the plurality of visit embeddings comprises processing, by a visit aggregator 306 of the longitudinal data processing component 202, each of the plurality of visit embeddings as an individual token. By way of non-limiting example, the visit aggregator 306 may comprise a transformer encoder. In some embodiments, the visit aggregator 306 incorporates fixed sine and cosine positional encodings as temporal encodings, processes the visit embeddings using a self-attention block, and employs average sequence pooling to generate the history embedding.

In some embodiments, processing the plurality of visit embeddings to generate the history embedding comprises projecting each patient visit's timestamp into a latent space using a linear transformation, and summing projected visit embeddings and temporal encodings. In some embodiments, processing the plurality of visit embeddings further comprises processing, using a transformer encoder 408, the plurality of individual tokens using self-attention mechanisms to capture both short-term and long-term dependencies among the plurality of patient visits, and applying, using a sequence pooling layer, average pooling along a token axis, aggregating information from the individual tokens into the history embedding, wherein the history embedding comprises a fixed-length representation.

In some embodiments, the longitudinal risk neural network 200 comprises a transformer-based sequence-to-class neural network, wherein each of the plurality of patient visits is encoded as an individual token.

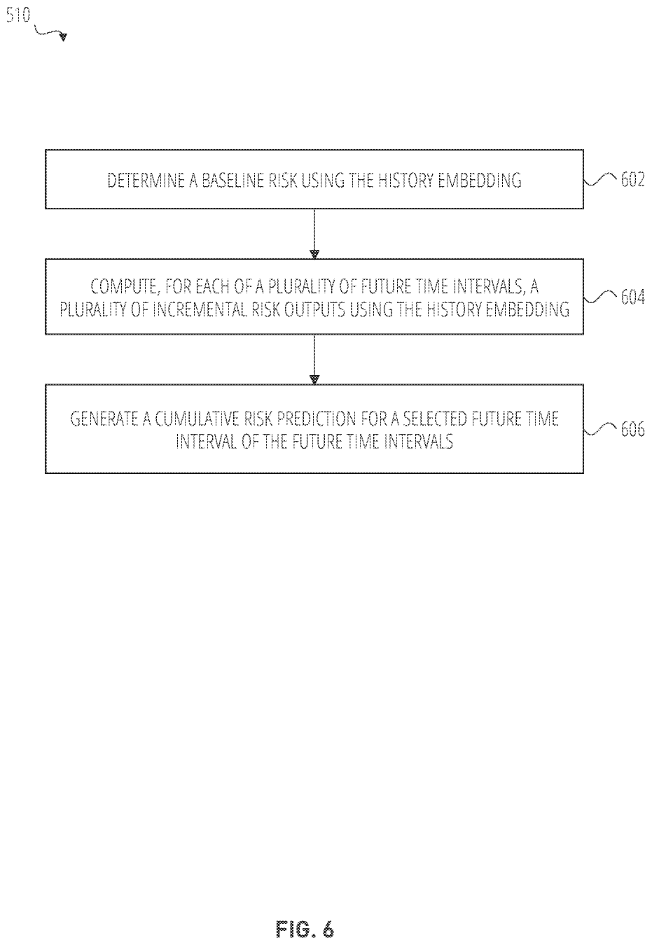

At operation 510, the processor generates, using the prediction component 204 of the longitudinal risk neural network 200, a clinical prediction based on the history embedding. Generating the clinical prediction comprises a plurality of sub-operations, as further described with reference to FIG. 6.

In some embodiments, the process 500 further comprises training the longitudinal risk neural network 200 as a single unified network on training data incorporating labels from a plurality of follow-up time intervals. In some embodiments, training the longitudinal risk neural network 200 comprises applying a data augmentation strategy in which one or more past patient visits are randomly omitted from a training input sequence to encourage generalization across varying amounts of longitudinal history.

In some embodiments, the process 500 further comprises outputting the clinical prediction to a user interface of a client device, such as the client device 102 described with reference to FIG. 1. In some embodiments, the process 500 further comprises providing one or more visual explanations of the clinical prediction, such as feature saliency maps or feature attributions, to guide interpretation of the clinical prediction.

FIG. 6 is a flowchart illustrating sub-operations of operation 510 of the process 500 described with reference to FIG. 5, in accordance with some examples. In particular, FIG. 6 illustrates sub-operations performed by the prediction component 204 of the longitudinal risk neural network 200 to generate the clinical prediction based on the history embedding. Although the sub-operations of operation 510 are described with reference to a particular order, the sub-operations may be performed in any suitable order, and one or more sub-operations may be performed sequentially, in parallel, or in any combination thereof.

The prediction component 204 receives the history embedding generated by the longitudinal data processing component 202 as described with reference to FIG. 5, and processes the history embedding to generate the clinical prediction.

At operation 602, the processor determines a baseline risk using the history embedding. In some embodiments, the baseline risk is determined by processing the history embedding through one or more linear layers of the prediction component 204 to produce a baseline risk value representative of a risk associated with a current time interval. By way of non-limiting example, the baseline risk may be denoted as B(m), where m represents the history embedding. The baseline risk represents a starting point from which incremental risk contributions associated with future time intervals are accumulated.

At operation 604, the processor computes, for each of a plurality of future time intervals, a plurality of incremental risk outputs using the history embedding. In some embodiments, the plurality of future time intervals corresponds to a plurality of follow-up time horizons extending from a current time interval into the future. By way of non-limiting example, the plurality of future time intervals may comprise one or more of a one-year time interval, a two-year time interval, a three-year time interval, a four-year time interval, a five-year time interval, or any other suitable time interval.

In some embodiments, computing the plurality of incremental risk outputs comprises processing the history embedding through a plurality of linear layers of the prediction component 204 to produce an incremental risk output for each of the plurality of future time intervals. The incremental risk outputs are passed through Rectified Linear Unit (ReLU) activations, such that the incremental risk outputs are constrained to be non-negative. By way of non-limiting example, the incremental risk output associated with an i-th future time interval may be denoted as Hᵢ(m), where m represents the history embedding. The non-negativity constraint on the incremental risk outputs ensures that a cumulative risk prediction generated from the incremental risk outputs is monotonically non-decreasing across the plurality of future time intervals.

At operation 606, the processor generates a cumulative risk prediction for a selected future time interval of the plurality of future time intervals. The cumulative risk prediction is generated by combining the baseline risk with a sum of the incremental risk outputs associated with a current time interval to the incremental risk outputs associated with the selected future time interval. In some embodiments, generating the cumulative risk prediction comprises computing a sum of the baseline risk and the incremental risk outputs from the current time interval up to the selected future time interval, and applying a sigmoid activation function to the sum to produce the cumulative risk prediction as a probability value.

By way of non-limiting example, the cumulative risk prediction P(t=k|m) for the selected future time interval k may be expressed as:

$$P ( t = k \vert m ) = σ ( B ( m ) + {\sum }_{i = 1}^{k} {H}_{i} ( m ) )$$

where σ denotes the sigmoid activation function, B(m) denotes the baseline risk determined at operation 602, Hᵢ(m), denotes the incremental risk output associated with the i-th future time interval computed at operation 604, and k denotes the selected future time interval. Because each Hᵢ(m) is constrained to be non-negative by the ReLU activation, the cumulative risk prediction P(t=k|m) is monotonically non-decreasing as k increases.

In some embodiments, the prediction component 204 generates a cumulative risk prediction for each of the plurality of future time intervals, thereby producing a sequence of cumulative risk predictions representing predicted risk across the plurality of future time intervals. In such embodiments, the sequence of cumulative risk predictions represents an ordinal prediction of risk progression over time, and the monotonic non-decrease property ensures that predicted risk at a later time interval is not less than predicted risk at an earlier time interval.

In some embodiments, generating the clinical prediction based on the cumulative risk prediction comprises generating the clinical prediction as a likelihood of disease or cancer based on the cumulative risk prediction, or generating the clinical prediction as a categorization of a condition based on the cumulative risk prediction. By way of non-limiting example, where the medical data comprises medical imaging data including mammograms, the clinical prediction may comprise a likelihood of breast cancer or a risk of breast cancer at the selected future time interval. By way of further non-limiting example, where the medical data comprises cognitive assessments, MRI volumetric measurements, and CSF biomarkers, the clinical prediction may comprise a likelihood of progression from a cognitively normal state to a mild cognitive impairment state, or a likelihood of progression from the mild cognitive impairment state to an Alzheimer's disease state, at the selected future time interval.

In some embodiments, the prediction component 204 comprises a first prediction component configured to perform operations 602, 604, and 606 for predicting a first level of progression, and a second prediction component configured to perform operations 602, 604, and 606 for predicting a second level of progression. In such embodiments, the first prediction component and the second prediction component share a common longitudinal data processing component 202 such that the history embedding generated by the longitudinal data processing component 202 is provided to both the first prediction component and the second prediction component. Generating the clinical prediction comprises determining whether the patient is associated with the first level of progression or the second level of progression, and selecting the cumulative risk prediction generated by the first prediction component or the cumulative risk prediction generated by the second prediction component based on the determination. By way of non-limiting example, the first level of progression may comprise a transition from a cognitively normal state to a mild cognitive impairment state, and the second level of progression may comprise a transition from the mild cognitive impairment state to an Alzheimer's disease state.

In some embodiments, the plurality of future time intervals comprises five annual time intervals extending from a current time interval, such that operation 604 computes five incremental risk outputs and operation 606 generates up to five cumulative risk predictions corresponding to one-year, two-year, three-year, four-year, and five-year future time horizons. In some embodiments, the plurality of future time intervals comprises time intervals corresponding to months, quarters, years, or any other suitable temporal granularity.

In some embodiments, the method further comprises outputting the cumulative risk prediction for the selected future time interval to a user interface of a client device, such as the client device 102 described with reference to FIG. 1. In some embodiments, the method further comprises outputting the sequence of cumulative risk predictions across the plurality of future time intervals to enable visualization of a predicted risk trajectory for the patient over time.

Existing artificial intelligence and machine learning systems for medical data analysis present a number of technical problems that limit their clinical utility. Many conventional systems rely on data from a single patient visit, or from one or two recent visits, and fail to capture disease-relevant patterns that emerge across a patient's longitudinal record. Conventional rule-based risk models rely on a limited number of manually selected variables and do not exploit the richness of longitudinal imaging data, multi-modal biomarkers, or repeated clinical measurements.

Embodiments of the present disclosure provide technical solutions to the foregoing technical problems by providing a longitudinal risk neural network architecture that integrates data from an arbitrary number of patient visits and a plurality of data modalities into a unified, time-aware predictive framework. The longitudinal data processing component combines a modality-specific encoder, an image aggregator, and a visit aggregator implemented using transformer encoders to generate a fixed-length history embedding from variable-length input sequences and irregular visit intervals. A knockout layer assigns out-of-distribution or highly improbable values to missing features, enabling the longitudinal risk neural network to process patient visits having incomplete data modalities. A temporal encoding mechanism based on a time interval between a patient visit's data collection and a target future prediction time captures both short-term and long-term dependencies among patient visits. The prediction component implements an additive hazard formulation in which incremental risk outputs are constrained to be non-negative by Rectified Linear Unit activations, such that a cumulative risk prediction is monotonically non-decreasing across a plurality of future time intervals, producing ordinal risk trajectories from a single unified network. Optional dual prediction components enable stage-conditional risk prediction across distinct disease progression transitions using a shared longitudinal data processing component.

These technical solutions provide a novel improvement over the current state of the art by enabling accurate and flexible clinical prediction across a range of disease domains and data modalities, including oncology (for example, breast cancer risk prediction from longitudinal mammograms), neurology (for example, Alzheimer's disease progression prediction from longitudinal cognitive assessments, MRI volumetric measurements, and cerebrospinal fluid biomarkers), and cardiology. By accommodating arbitrary numbers of patient visits, variable data collection frequencies, missing features across data modalities, and multiple future prediction horizons within a single unified model, the longitudinal risk neural network improves predictive performance relative to single-timepoint and narrowly scoped longitudinal models and enables personalized screening and intervention strategies based on a patient's evolving longitudinal record.

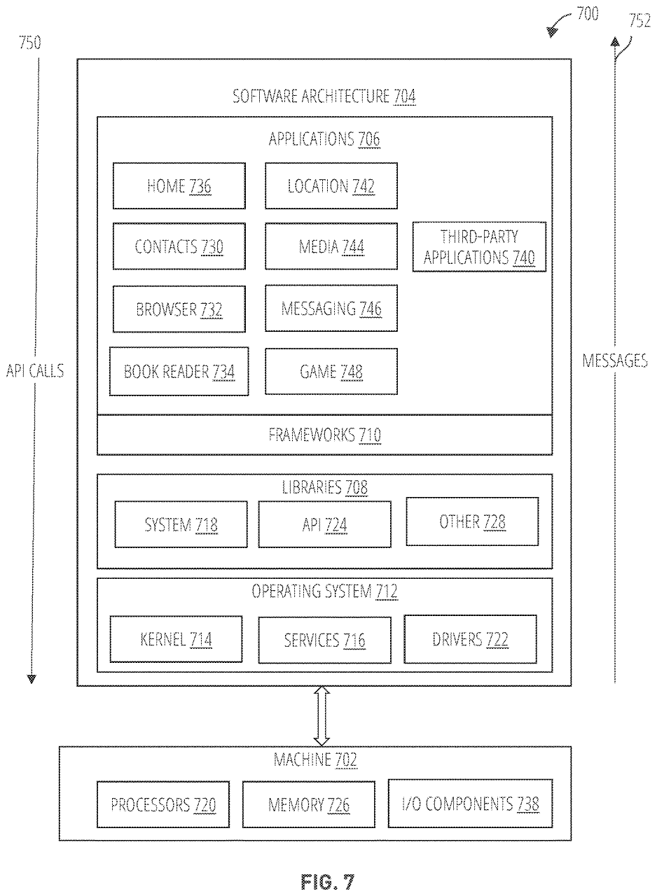

FIG. 7 is a block diagram 700 illustrating a software architecture 704, which can be installed on any one or more of the devices described herein. The software architecture 704 is supported by hardware such as a machine 702 that includes processors 720, memory 726, and I/O components 738. In this example, the software architecture 704 can be conceptualized as a stack of layers, where individual layers provide a particular functionality. The software architecture 704 includes layers such as an operating system 712, libraries 708, frameworks 710, and Applications 706. Operationally, the Applications 706 invoke API calls 750 through the software stack and receive messages 752 in response to the API calls 750.

The operating system 712 manages hardware resources and provides common services. The operating system 712 includes, for example, a kernel 714, services 716, and drivers 722. The kernel 714 acts as an abstraction layer between the hardware and the other software layers. For example, the kernel 714 provides memory management, processor management (e.g., scheduling), component management, networking, and security settings, among other functionalities. The services 716 can provide other common services for the other software layers. The drivers 722 are responsible for controlling or interfacing with the underlying hardware. For instance, the drivers 722 can include display drivers, camera drivers, BLUETOOTH® or BLUETOOTH® Low Energy drivers, flash memory drivers, serial communication drivers (e.g., Universal Serial Bus (USB) drivers), WI-FI® drivers, audio drivers, power management drivers, and so forth.

The libraries 708 provide a low-level common infrastructure used by the Applications 706. The libraries 708 can include system libraries 718 (e.g., C standard library) that provide functions such as memory allocation functions, string manipulation functions, mathematical functions, and the like. In addition, the libraries 708 can include API libraries 724 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) graphic content on a display, GLMotif used to implement 3D user interfaces), image feature extraction libraries (e.g. OpenIMAJ), database libraries (e.g., SQLite to provide various relational database functions), web libraries (e.g., WebKit to provide web browsing functionality), and the like. The libraries 708 can also include a wide variety of other libraries 728 to provide many other APIs to the Applications 706.

The frameworks 710 provide a high-level common infrastructure that is used by the Applications 706. For example, the frameworks 710 provide various graphical user interface (GUI) functions, high-level resource management, and high-level location services. The frameworks 710 can provide a broad spectrum of other APIs that can be used by the Applications 706, some of which may be specific to a particular operating system or platform.

In an example, the Applications 706 may include a home Application 736, a contacts Application 730, a browser Application 732, a book reader Application 734, a location Application 742, a media Application 744, a messaging Application 746, a game Application 748, and a broad assortment of other Applications such as third-party Applications 740. The Applications 706 are programs that execute functions defined in the programs. Various programming languages can be employed to create one or more of the Applications 706, structured in a variety of manners, such as object-oriented programming languages (e.g., Objective-C, Java, or C++) or procedural programming languages (e.g., C or assembly language). In a specific example, the third-party Applications 740 (e.g., Applications developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party Applications 740 can invoke the API calls 750 provided by the operating system 712 to facilitate functionality described herein.

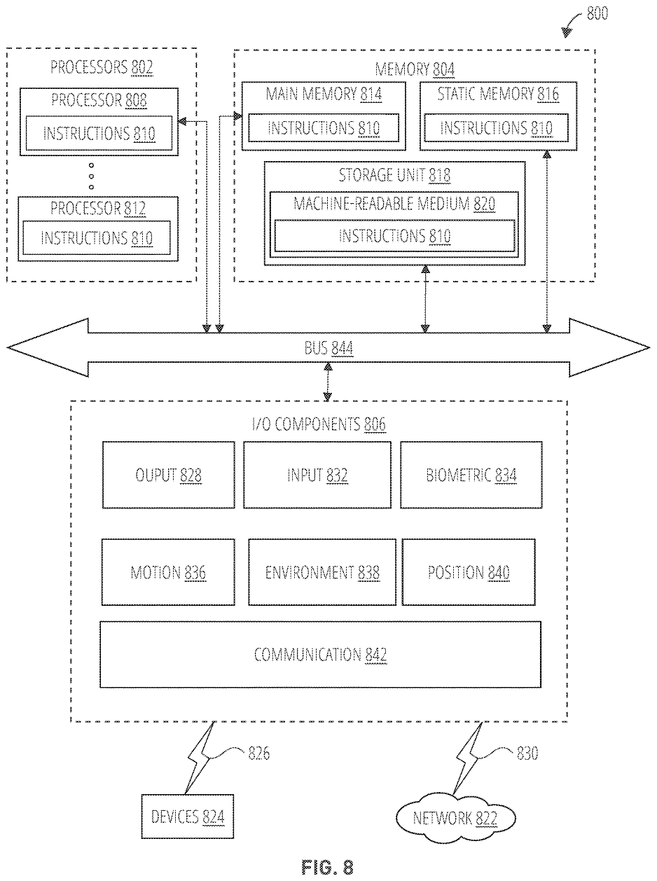

FIG. 8 is a diagrammatic representation of a machine 800 within which instructions 810 (e.g., software, a program, an application, an applet, an app, or other executable code) for causing the machine 800 to perform any one or more of the methodologies discussed herein may be executed. For example, the instructions 810 may cause the machine 800 to execute any one or more of the methods described herein. The instructions 810 transform the general, non-programmed machine 800 into a particular machine 800 programmed to carry out the described and illustrated functions in the manner described. The machine 800 may operate as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 810, sequentially or otherwise, that specify actions to be taken by the machine 800. Further, while a single machine 800 is illustrated, the term “machine” may also be taken to include a collection of machines that individually or jointly execute the instructions 810 to perform any one or more of the methodologies discussed herein.

The machine 800 may include processors 802, memory 804, and I/O components 806, which may be configured to communicate with one another via a bus 844. In an example, the processors 802 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 808 and a processor 812 that execute the instructions 810. The term “processor” is intended to include multi-core processors that may comprise two or more independent processors (sometimes referred to as “cores”) that may execute instructions contemporaneously. Although FIG. 8 shows multiple processors 802, the machine 800 may include a single processor with a single core, a single processor with multiple cores (e.g., a multi-core processor), multiple processors with a single core, multiple processors with multiple cores, or any combination thereof.

The memory 804 includes a main memory 814, a static memory 816, and a storage unit 818, both accessible to the processors 802 via the bus 844. The main memory 814, the static memory 816, and storage unit 818 store the instructions 810 embodying any one or more of the methodologies or functions described herein. The instructions 810 may also reside, completely or partially, within the main memory 814, within the static memory 816, within machine-readable medium 820 within the storage unit 818, within one or more of the processors 802 (e.g., within the processor's cache memory), or any suitable combination thereof, during execution thereof by the networked system 100.

The I/O components 806 may include a wide variety of components to receive input, provide output, produce output, transmit information, exchange information, capture measurements, and so on. The specific I/O components 806 that are included in a particular machine will depend on the type of machine. For example, portable machines such as mobile phones may include a touch input device or other such input mechanisms, while a headless server machine will likely not include such a touch input device. It will be appreciated that the I/O components 806 may include many other components that are not shown in FIG. 8. In various examples, the I/O components 806 may include output components 828 and input components 832. The output components 828 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 832 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further examples, the I/O components 806 may include biometric components 834, motion components 836, environmental components 838, or position components 840, among a wide array of other components. For example, the biometric components 834 include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 836 include acceleration sensor components (e.g., accelerometer), gravitation sensor components, rotation sensor components (e.g., gyroscope), and so forth. The environmental components 838 include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 840 include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 806 further include communication components 842 operable to couple the networked system 100 to a network 822 or devices 824 via a coupling 830 and a coupling 826, respectively. For example, the communication components 842 may include a network interface component or another suitable device to interface with the network 822. In further examples, the communication components 842 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 824 may be another machine or any of a wide variety of peripheral devices (e.g., a peripheral device coupled via a USB).

Moreover, the communication components 842 may detect identifiers or include components operable to detect identifiers. For example, the communication components 842 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 842, such as location via Internet Protocol (IP) geolocation, location via Wi-Fi® signal triangulation, location via detecting an NFC beacon signal that may indicate a particular location, and so forth.

The various memories (e.g., memory 804, main memory 814, static memory 816, and/or memory of the processors 802) and/or storage unit 818 may store one or more sets of instructions and data structures (e.g., software) embodying or used by any one or more of the methodologies or functions described herein. These instructions (e.g., the instructions 810), when executed by processors 802, cause various operations to implement the disclosed examples.

The instructions 810 may be transmitted or received over the network 822, using a transmission medium, via a network interface device (e.g., a network interface component included in the communication components 842) and using any one of a number of well-known transfer protocols (e.g., hypertext transfer protocol (HTTP)). Similarly, the instructions 810 may be transmitted or received using a transmission medium via the coupling 826 (e.g., a peer-to-peer coupling) to the devices 824.

### GLOSSARY

A “carrier signal” refers to any intangible medium that is capable of storing, encoding, or carrying instructions for execution by the machine, and includes digital or analog communications signals or other intangible media to facilitate communication of such instructions. Instructions may be transmitted or received over a network using a transmission medium via a network interface device.

A “client device” refers to any machine that interfaces to a communications network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network.

A “communication network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long-range protocols, or other data transfer technology.

A “component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing some operations and may be configured or arranged in a particular physical manner. In various examples, one or more computer systems (e.g., a standalone computer system, a client computer system, or a server computer system) or one or more hardware components of a computer system (e.g., a processor or a group of processors) may be configured by software (e.g., an Application or Application portion) as a hardware component that operates to perform some operations as described herein. A hardware component may also be implemented mechanically, electronically, or any suitable combination thereof. For example, a hardware component may include dedicated circuitry or logic that is permanently configured to perform some operations. A hardware component may be a special-purpose processor, such as a field-programmable gate array (FPGA) or an application specific integrated circuit (ASIC). A hardware component may also include programmable logic or circuitry that is temporarily configured by software to perform some operations. For example, a hardware component may include software executed by a general-purpose processor or other programmable processor. Once configured by such software, hardware components become specific machines (or specific components of a machine) tailored to perform the configured functions and are no longer general-purpose processors. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component” (or “hardware-implemented component”) is to be understood to encompass a tangible entity, be that an entity that is physically constructed, permanently configured (e.g., hardwired), or temporarily configured (e.g., programmed) to operate in a particular manner or to perform some operations described herein. Considering examples in which hardware components are temporarily configured (e.g., programmed), the hardware components may not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In examples in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be partially processor-implemented, with a particular processor or processors being an example of hardware. For example, some of the operations of a method may be performed by one or more processors or processor-implemented components. Moreover, the one or more processors may also operate to support performance of the relevant operations in a “cloud computing” environment or as a “software as a service” (SaaS). For example, some of the operations may be performed by a group of computers (as examples of machines including processors), with these operations being accessible via a network (e.g., the Internet) and via one or more appropriate interfaces (e.g., an API). The performance of some of the operations may be distributed among the processors, residing within a single machine as well as being deployed across a number of machines. In some examples, the processors or processor-implemented components may be located in a single geographic location (e.g., within a home environment, an office environment, or a server farm). In other examples, the processors or processor-implemented components may be distributed across a number of geographic locations.

A “computer-readable medium” refers to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

A “machine-storage medium” refers to a single or multiple storage devices and/or media (e.g., a centralized or distributed database, and/or associated caches and servers) that store executable instructions, routines and/or data. The term includes, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, some of which are covered under the term “signal medium.”

A “processor” refers to any circuit or virtual circuit (a physical circuit emulated by logic executing on an actual processor) that manipulates data values according to control signals (e.g., “commands”, “op codes”, “machine code”, and so forth) and which produces corresponding output signals that are applied to operate a machine. A processor may, for example, be a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC) or any combination thereof. A processor may further be a multi-core processor having two or more independent processors (sometimes referred to as “cores”) that may execute instructions contemporaneously.

A “signal medium” refers to any intangible medium that is capable of storing, encoding, or carrying the instructions for execution by a machine and includes digital or analog communications signals or other intangible media to facilitate communication of software or data. The term “signal medium” may be taken to include any form of a modulated data signal, carrier wave, and so forth. The term “modulated data signal” means a signal that has one or more of its characteristics set or changed in such a manner as to encode information in the signal. The terms “transmission medium” and “signal medium” mean the same thing and may be used interchangeably in this disclosure.

Changes and modifications may be made to the disclosed examples without departing from the scope of the present disclosure. These and other changes or modifications are intended to be included within the scope of the present disclosure, as expressed in the following claims.

What is claimed is:

1. A method for clinical predictions based on longitudinal medical data analysis, comprising:
receiving, by a processor, using longitudinal data processing component of a longitudinal risk neural network, medical data from a plurality of modalities associated with a plurality of patient visits over time, wherein the patient visits are associated with a patient;
encoding the medical data to generate encoded medical data;
generating a plurality of visit embeddings based on the encoded medical data of each of patient visit of the plurality of patient visits;
processing the plurality of visit embeddings to capture temporal relationships between the visit embeddings, and to generate a history embedding representing a longitudinal record for the patient; and
generating, using a prediction component of the longitudinal risk neural network, a clinical prediction based on the history embedding, wherein generating the clinical prediction comprises:
determining a baseline risk using the history embedding;
computing, for each of a plurality of future time intervals, a plurality of incremental risk outputs using the history embedding, wherein the incremental risk outputs are constrained to be non-negative; and
generating a cumulative risk prediction for a selected future time interval of the future time intervals by combining the baseline risk with a sum of the incremental risk outputs associated with a current time interval to the incremental risk outputs associated with the selected future time interval.

2. The method of claim 1, wherein generating the clinical prediction further comprises:
computing, for each of a plurality of future time intervals, the plurality of incremental risk outputs using the history embedding and linear layers;
passing the incremental risk outputs through Rectified Linear Unit (ReLU) activations, wherein the incremental risk outputs are constrained to be non-negative.

3. The method of claim 1, wherein the medical data from a plurality of modalities comprise medical imaging data, risk factor metadata, laboratory values, clinical test results, clinical notes, waveform data genetic data, other data types, or any combination thereof,
wherein the medical imaging data comprises mammograms, ultrasound images, X-ray images, Magnetic Resonance Imaging (MRI) images, computed tomography (CT) scans, or any combination thereof, and
wherein the risk factor metadata comprises age, family history, BMI, hormonal factors, or any combination thereof.

4. The method of claim 1, wherein the medical data from a plurality of modalities comprise
cognitive assessments, cerebrospinal fluid (CSF) biomarkers, MRI volumetric measurements,
genetic data, demographic data, or any combination thereof,
wherein the demographic data comprises age, sex, education, marital status, race, ethnicity, or any combination thereof.

5. The method of claim 1, wherein encoding the medical data to generate the encoded medical data further comprises:
processing, by an image encoder of the longitudinal data processing component, the medical data to generate a plurality of image embeddings associated with the plurality patient visits,
wherein the encoded medical data of each of patient visit of the plurality of patient visits comprises the plurality of image embeddings associated with the plurality patient visits.

6. The method of claim 5, wherein generating the plurality of visit embeddings further comprises:
combining, by an image aggregator of the longitudinal data processing component, the plurality of image embeddings to generate the plurality of visit embeddings.

7. The method of claim 6, wherein processing the plurality of visit embeddings to capture temporal relationships between the visit embeddings, and to generate a history embedding representing the longitudinal record of the patient further comprises:
processing, by a visit aggregator of the longitudinal data processing component, each of the plurality of visit embeddings as an individual token.

8. The method of claim 7, wherein
the image encoder comprises a Residual Network (ResNet)-based convolutional neural network (CNN);
the image aggregator and the visit aggregator comprise transformer encoders.

9. The method of claim 8, wherein the visit aggregator incorporates fixed sine and cosine positional encodings as temporal encodings, wherein the visit aggregator processes using a self-attention block and employing average sequence pooling to generate the history embedding.

10. The method of claim 1, wherein generating, using the prediction component, the clinical prediction based on the history embedding further comprises:
generating the clinical prediction being a likelihood of disease or cancer based on the cumulative risk prediction, or being a categorization of a condition based on the cumulative risk prediction.

11. The method of claim 10,
wherein the medical data comprises medical imaging data including mammograms, X-ray scans from a craniocaudal (CC), mediolateral oblique (MLO) views for both left and right breasts, or any combination thereof, and
wherein the clinical prediction is the likelihood of breast cancer or risk of breast cancer.

12. The method of claim 1, wherein the longitudinal risk neural network comprises a Transformer-based sequence-to-class neural network, wherein each of the plurality of patient visits is encoded as an individual token.

13. The method of claim 1, wherein encoding the medical data to generate encoded medical data further comprises:
processing, by a knockout layer, for each of the patient visits, the medical data to identify missing features; and
assigning, by the knockout layer, out-of-distribution or highly improbable values to the missing features.

14. The method of claim 13, wherein generating a plurality of visit embeddings based on the encoded medical data of each of patient visit of the plurality of patient visits further comprises:
encoding temporal ordering of the plurality of patient visits by:
incorporating temporal encoding vectors within each of a plurality of individual tokens, wherein the plurality of patient visits are encoded as the plurality of individual tokens, wherein the temporal encoding vectors are based on the time interval between a patient visit's data collection and a target future prediction time.

15. The method of claim 14, wherein processing the plurality of visit embeddings to capture temporal relationships between the visit embeddings and to generate the history embedding representing the longitudinal record of the patient further comprises:
projecting each patient visit's timestamp into a latent space using a linear transformation; and
summing projected visit embeddings and temporal encodings.

16. The method of claim 15, wherein processing the plurality of visit embeddings to capture temporal relationships between the visit embeddings and to generate the history embedding representing the longitudinal record of the patient further comprises:
processing, using a transformer encoder, the plurality of individual tokens using self-attention mechanisms to capture both short-term and long-term dependencies among the plurality of patient visits; and
applying, using a sequence pooling layer, average pooling along a token axis, aggregating information from the individual tokens into the history embedding, wherein the history embedding comprises a fixed-length representation.

17. The method of claim 16, wherein the prediction component comprises a first prediction component for predicting a first level of progression and a second prediction component for predicting a second level of progression, and wherein generating the clinical prediction comprises:
determining whether the patient is associated with the first level of progression or the second level of progression;
generating the clinical prediction using the first prediction component when the patient is determined to be associated with the first level of progression or using the second prediction component when the patient is determined to be associated with the second level of progression.

18. The method of claim 17, wherein the first level of progression comprises a transition from a cognitively normal state to a mild cognitive impairment state, and the second level of progression comprises a transition from the mild cognitive impairment state to an Alzheimer's disease state.

19. A system for clinical predictions based on longitudinal medical data analysis, comprising:
one or more processors; and
memory storing instructions that, when executed by the one or more processors, cause the system to:
receive, using a longitudinal data processing component of a longitudinal risk neural network, medical data from a plurality of modalities associated with a plurality of patient visits over time, wherein the patient visits are associated with a patient;
encode the medical data to generate encoded medical data;
generate a plurality of visit embeddings based on the encoded medical data of each patient visit of the plurality of patient visits;
process the plurality of visit embeddings to capture temporal relationships between the visit embeddings and to generate a history embedding representing a longitudinal record for the patient; and
generate, using a prediction component of the longitudinal risk neural network, a clinical prediction based on the history embedding, wherein generating the clinical prediction comprises:
determining a baseline risk using the history embedding;
computing, for each of a plurality of future time intervals, a plurality of incremental risk outputs using the history embedding; and
generating a cumulative risk prediction for a selected future time interval of the future time intervals by combining the baseline risk with a sum of the incremental risk outputs associated with a current time interval to the incremental risk outputs associated with the selected future time interval.

20. A non-transitory computer-readable medium storing instructions that, when executed by one or more processors, cause the one or more processors to perform operations comprising:
receiving, using a longitudinal data processing component of a longitudinal risk neural network, medical data from a plurality of modalities associated with a plurality of patient visits over time, wherein the patient visits are associated with a patient;
encoding the medical data to generate encoded medical data;
generating a plurality of visit embeddings based on the encoded medical data of each patient visit of the plurality of patient visits;
processing the plurality of visit embeddings to capture temporal relationships between the visit embeddings and to generate a history embedding representing a longitudinal record for the patient; and
generating, using a prediction component of the longitudinal risk neural network, a clinical prediction based on the history embedding, wherein generating the clinical prediction comprises:
determining a baseline risk using the history embedding;
computing, for each of a plurality of future time intervals, a plurality of incremental risk outputs using the history embedding; and
generating a cumulative risk prediction for a selected future time interval of the future time intervals by combining the baseline risk with a sum of the incremental risk outputs associated with a current time interval to the incremental risk outputs associated with the selected future time interval.
